(19) United States
(12) Patent Application Publication
(10) Pub. No.: US 2022/0117823 A1
(43) Pub. Date: Apr. 21, 2022

(54) Patient-Responsive Orthosis

(71) Applicant: MAR Orthotics, Inc., Milwaukee, WI (US)

(72) Inventors: Michael J. Sobrepera, Philadelphia, PA (US); Ariella Mansfield, Philadelphia, PA (US); Rebecca M. Li, Philadelphia, PA (US)

(21) Appl. No.: 17/073,755

(22) Filed: Oct. 19, 2020

(51) Int. Cl. A61H 1/02; A61F 5/01

(52) U.S. Cl. CPC A61H 1/0285; A61F 5/013; A61H 2201/1635; A61B 5/4809; A61H 2201/501; A61H 2201/5064; A61H 2201/165

(57) ABSTRACT

Orthoses, which are recommended for those suffering from spastic cerebral palsy to wear during sleep, may require manual adjustment and can cause discomfort while the wearer is trying to fall asleep. Accordingly, a patient-responsive orthosis is provided that includes a forearm splint, a hand actuator, a motor that extends the hand of the wearer by moving the hand actuator, memory that stores instructions, and a processor that extends the hand of the wearer in response to the instructions by causing the motor to move the hand actuator. The orthosis may include a sleep sensor and may be programmed to extend the hand of the wearer while the wearer is asleep. The patient-responsive orthosis may be programmed (e.g., by a clinician specifying a treatment schedule) using a remote device. The patient-responsive orthosis may also store and output usage data (e.g., for review by a clinician).

### CROSS-REFERENCE TO RELATED APPLICATIONS

None

### FEDERAL FUNDING

None

### BACKGROUND

Cerebral palsy (CP) is the most common motor disorder affecting young children. The most common form of cerebral palsy is spastic cerebral palsy, which leads to spasticity, an involuntary contracture of muscles. In order to treat spasticity, a combination of stretching with an orthosis and Botox therapy is generally used. (BOTOX is a registered trademark of Allergan Inc.) An orthosis is an orthotic device that may include a splint, a brace, a wrap or a support. An orthosis may be a rigid or semi-rigid device that supports a weak or deformed body member or restricts or eliminates motion in a diseased or injured part of the body. An orthosis can be custom fabricated, custom fit, or prefabricated. An orthosis is often indicated for wear during sleep to provide muscle extension without affecting day-to-day activities.

Prior art orthoses are either rigid, manually adjustable by a clinician, or apply constant force using springs. FIG. 1 illustrates a prior art static orthosis 100. The static orthosis 100 is molded once for the user and therefore needs to be replaced when treatment progresses. In addition, the static orthosis 100 can cause painful pressure sores and may lead to incorrect usage or a patient discontinuing treatment to avoid the discomfort. FIG. 2 illustrates a prior art passive spring orthosis 200. The passive spring orthosis 200 is slightly flexible. However, passive spring orthosis 200 requires manual adjustment by a clinician. Additionally, applying constant spring force may be harmful for spasticity.

Wearing the prior art static orthosis 100 or the prior art passive spring orthosis 200 can be uncomfortable or even painful, often preventing the child from falling asleep. Children may resist wearing a prior art orthosis 100 or 200 due to discomfort, which can lead to emotional strife between parents and their children as the parents attempt to persuade a child to comply with their treatment while the child attempts to fall asleep. Failure to adhere to prescribed treatment can lead to further loss of function as well as disqualification for surgical treatments. Lack of treatment may lead to long term challenges in completing activities of daily living resulting in reduction in quality of life.

Accordingly, there is a need for a patient-responsive orthosis that allows patients (e.g., suffering from cerebral palsy, having suffered a stroke, etc.) to comfortably fall asleep while still complying with their prescribed treatment regimen. Additionally, there is a need for an orthosis that provides feedback to patients and caregivers regarding patient treatment.

### SUMMARY

In order to overcome those and other deficiencies in the prior art, a patient-responsive orthosis is provided. The patient-responsive orthosis includes a forearm splint, a hand actuator, a motor that extends the hand of the wearer by moving the hand actuator, memory that stores instructions, and a processor that extends the hand of the wearer in response to the instructions by causing the motor to move the hand actuator.

The patient-responsive orthosis may include a sleep sensor and may be programmed to extend the hand of the wearer while the wearer is asleep. By allowing the wearer to fall asleep comfortably, the patient-responsive orthosis improves the quality of life of the wearer and any caregiver for the wearer. Allowing the wearer to fall asleep comfortably may also improve patient adherence to the prescribed treatment, which may be implemented only as the wearer is sleeping. Better adherence to the treatment regimen may improve clinical outcomes for patients. Additionally, because lack of adherence to treatment can disqualify a patient from surgical treatments, the patient-responsive orthosis may help the wearer remain eligible for surgery.

The patient-responsive orthosis may be programmed (e.g., by a clinician) using a remote device. For instance, a clinician may specify when the wearer's hand is extended, how often the wearer's hand is extended, how gradually the wearer's hand is extended, the angle the hand actuator when the wearer's hand is extended, and the time period that the wearer's hand is extended before the orthosis returns to the relaxed position.

The patient-responsive orthosis may also store and output usage data (e.g., for review by a clinician). Accordingly, the patient-responsive orthosis enables a prescribed treatment regimen to be better tailored for the individual wearer. Usage data from multiple patient-responsive orthoses may even be aggregated and analyzed to study the efficacy of various treatments.

### BRIEF DESCRIPTION OF THE DRAWINGS

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the following drawings.

FIG. 1 illustrates a prior art static orthosis.

FIG. 2 illustrates a prior art passive spring orthosis.

FIG. 3A illustrates a patient-responsive orthosis in a relaxed position according to an exemplary embodiment of the present invention.

FIG. 3B illustrates the patient-responsive orthosis in an extended position according to an exemplary embodiment of the present invention.

FIG. 4 is a block diagram of a component package of the patient-responsive orthosis according to an exemplary embodiment of the present invention.

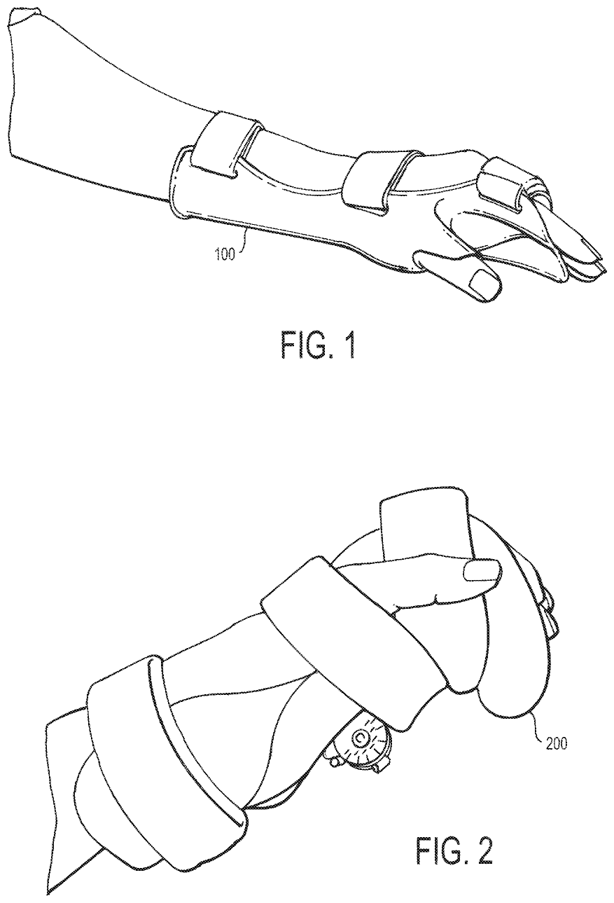

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

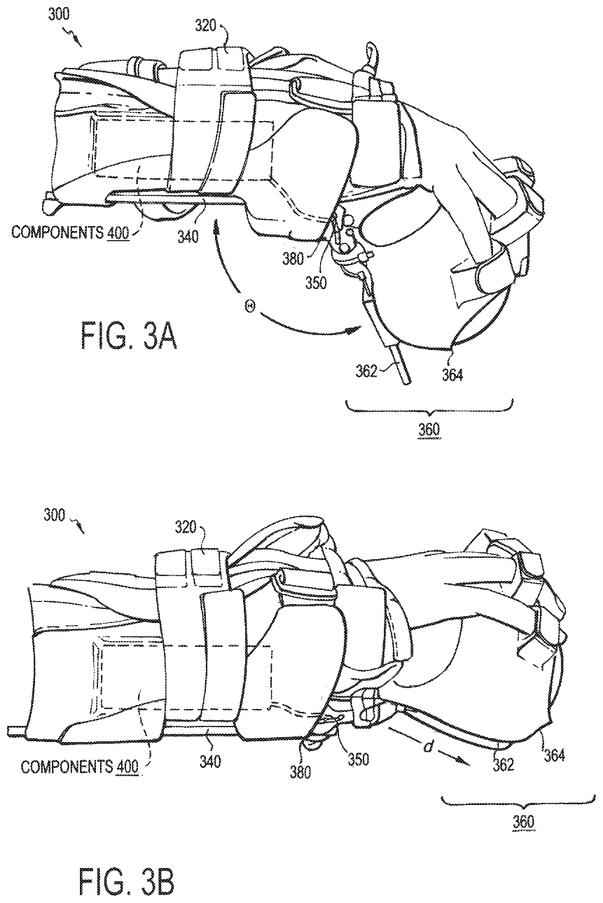

FIG. 3A illustrates a patient-responsive orthosis 300 in a relaxed position according to an exemplary embodiment of the present invention.

As shown in FIG. 3A, the patient-responsive orthosis 300 includes a fabric cuff 320, a forearm splint 340, a hand actuator 360, and a motor 380, which may be part of a component package 400.

The fabric cuff 320 secures the patient-responsive orthosis 300 to the forearm of a wearer. In some embodiments, the fabric cuff 320 may secure the patient-responsive orthosis 300 to the wrist and, in some embodiments, the base of one or more fingers of the wearer as well. The fabric cuff 320 may be attachable to the forearm of the wearer via straps with hook-and-loop fasteners, colloquially referred to as Velcro straps. (VELCRO is a registered trademark of Velcro Companies.)

The forearm splint 340 runs along the length of the forearm (e.g., under the forearm) of the wearer, provides structure for the patient-responsive orthosis 300, and provides a mounting point for the motor 380 and the other components included in the component package 400 (described below).

The motor 380 moves the hand actuator 360 relative to the forearm splint 340, to push the hand of the wearer into an outstretched position or allow the hand of the wearer to curl inwards into an unstretched position. The hand actuator 360 may be any device that is attachable to the fingers and/or hand of the wearer and enables the motor 380 to extend the fingers and/or hand of the wearer. In the embodiment shown in FIG. 3A, for example, the hand actuator 360 may include a hand splint 362 and an attachment mechanism 364. The hand splint 362 is connected to the forearm splint 340 via a hinge 350. The attachment mechanism 364 may be any suitable device that is attachable to the hand or fingers of the wearer. For example, in the embodiment shown, the attachment mechanism 364 is attachable to the fingers of the wearer via Velcro straps.

FIG. 3B illustrates the patient-responsive orthosis 300 in an extended position according to an exemplary embodiment of the present invention.

As shown in FIGS. 3A and 3B, the motor 380 moves the hand actuator 360 relative to the forearm splint 340. In the embodiment shown in FIGS. 3A and 3B, for example, the motor rotates the hand splint 362 about the hinge 350 and adjusts the angle θ of the hand splint 362 relative to the angle of the forearm splint 340. At the same time, the attachment mechanism 364 in the embodiment shown in FIGS. 3A and 3B moves in the direction d along the hand splint 362 away from the location of the forearm splint 340.

By moving the hand actuator 360 relative to the forearm splint 340 (in the embodiment shown, for example, rotating the hand splint 362 to increase the angle θ between the forearm splint 340 and the hand splint 362 and translating the attachment mechanism 364 in the direction d away from the forearm splint 340), the patient-responsive orthosis 300 extends the hand of the wearer into an outstretched position. Furthermore, by moving the hand actuator 360 toward the forearm splint 340 (in the embodiment shown, for example, rotating the hand splint 362 to decrease the angle θ between the forearm splint 340 and the hand splint 362 and allowing the attachment mechanism 364 to move along the hand splint 362 toward the forearm splint 340), the patient-responsive orthosis 300 extends allows the hand of the wearer to curl in a relaxed position.

In the embodiment shown in FIGS. 3A and 3B, the attachment mechanism 364 moves in the direction d along the hand splint 362. The attachment mechanism 364 moves passively, meaning the wearer's hand moves the attachment mechanism 364 along the hand splint 362 as the angle of the hand splint 362 is rotated. In other embodiments, the orthosis 300 may actively translate the attachment mechanism 364 along the hand splint 362 (e.g., by a motor). In still other embodiments, the attachment mechanism 364 may be affixed to the hand splint 362 such that it does not move along the hand splint 362.

In the embodiment shown in FIGS. 3A and 3B, the attachment mechanism 364 includes a ball that keeps the fingers of the wearer partially extended regardless of the angle of the hand splint 362. In other embodiments, the orthosis 300 may include finger actuators that extend fingers of the wearer. Those finger actuators may be extended by the motor 380 (or additional motors) and, similar to the hand actuator 360, may be controlled by the microcontroller 420 as described below.

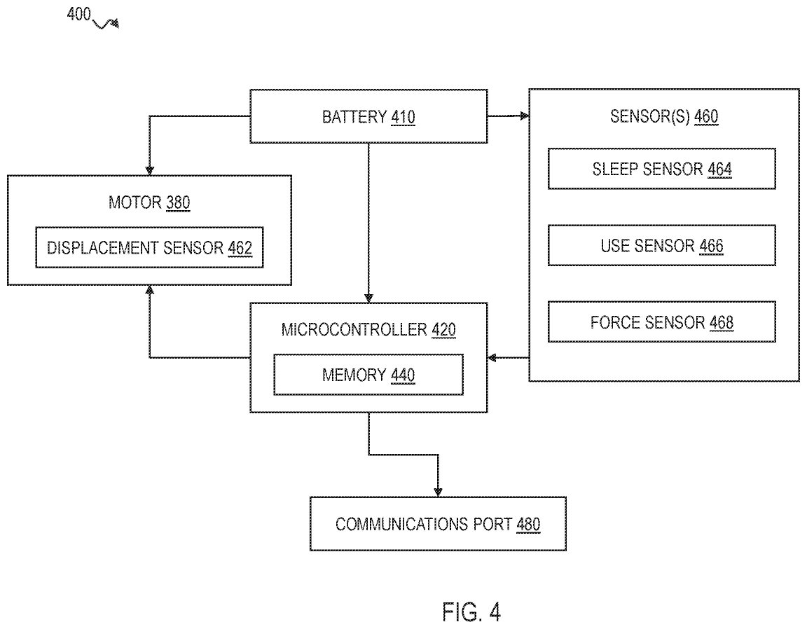

FIG. 4 is a block diagram of the component package 400 according to an exemplary embodiment of the present invention.

As shown in FIG. 4, the component package 400 includes a battery 410, a microcontroller 420, memory 440, and one or more sensors 460. The sensor(s) 460 include a displacement sensor 462 and may include a sleep sensor 464, a use sensor 466, and a force sensor 468. The component package 400 may also include a communications port 480.

The battery 410 provides power for the motor 380 and other components (such as the microcontroller 420 and the sensor(s) 460). The battery 410 may be rechargeable. Accordingly, the battery 410 may include a charging port. In some embodiments, the battery 410 may be removable from the patient-responsive orthosis 300 for convenient charging. In other embodiments, the battery 410 may remain attached to the patient-responsive orthosis 300 and may be recharged when the patient-responsive orthosis 300 is removed from the wearer.

The microcontroller 420 controls the patient-responsive orthosis 300. The microcontroller 420 may be any hardware computer processor capable of providing the functions described below. The microcontroller 420 may be, for example, an integrated circuit. In order to provide some of the functions described below, the microcontroller 420 includes a clocking mechanism, which may be integrated with the microcontroller 420 or a separate component in communication with the microcontroller 420.

The memory 440 may be any non-transitory computer readable storage media capable of storing the data described below and software instructions that, when executed by the microcontroller 420, provide the functionality described below. The memory 440 may be, for example, solid state memory.

The displacement sensor 462 measures the location of the hand actuator 360 relative to the forearm splint 340. In the embodiment shown in FIGS. 3A and 3B, for example, the displacement sensor 462 may be an angular displacement sensor that measures the angle θ of the hand splint 362 relative to the forearm splint 340 (or another starting position). In other embodiments, the displacement sensor 462 may measure linear displacement of the hand actuator 360 (for example, the linear displacement of the attachment mechanism 364 along the hand splint 362).

The displacement sensor 462 may be an optical encoder, a magnetic encoder, a potentiometer, etc. In some embodiments, the displacement sensor 462 may be an angular displacement sensor that is integrated with the motor 360. In those embodiments, the angular displacement sensor measures the angle of the motor 360 relative to an initial starting position. In the embodiment shown in FIG. 4, the displacement sensor 462 may be integrated with the motor 360. An angular displacement sensor that is integrated with the motor 360 may also measure the torque on the wrist of the wearer. In other embodiments, the displacement sensor 462 may be a discrete component that is separate from the motor 360.

The patient-responsive orthosis 300 may optionally include a sleep sensor 464 that outputs an indication to the microcontroller 420 that the wearer is asleep. The sleep sensor 464 may be any suitable device capable of determining if the wearer is asleep, for example based on reduced motion of the wearer (for example, as determined by a motion sensor), a decrease in skin temperature (for example, as determined by a skin temperature thermometer), galvanic skin response data (for example, as determined by an electrodermal activity sensor), reduced heart rate (for example, as determined by a pulse monitor), reduced breathing rate (for example, as determined by a microphone recording breathing sounds), etc.

In the embodiment shown in FIG. 4, the patient-responsive orthosis 300 includes an integrated sleep sensor 464. In other embodiments, however, an indication that the wearer is asleep may be received by the microcontroller 420 from an external device, such as a wearable activity tracker (as described, for example, in U.S. Pat. Pub. No. 2015/0250396 to Ahmed, et al) or a mattress or bed configured to determine the sleep state of a user lying on the mattress or bed (as described, for example, in U.S. Pat. Pub. No. 2014/0259434 to Nunn, et al.). In embodiments where the microcontroller 420 is configured to receive an indication that the wearer is asleep from an external device, the communications port 480 (described below) may include a wireless communications module to receive that indication.

The patient-responsive orthosis 300 may include a use sensor 466 that outputs an indication to the microcontroller 420 that wearer is wearing the orthosis 300. The use sensor 466 may be any suitable device capable of determining the presence of the wearer. The use sensor 466 may be, for example, a contact switch, a proximity sensor, a motion sensor, a skin temperature thermometer, an electrodermal activity sensor, a pulse monitor, etc. In embodiments that include both a sleep sensor 464 and use sensor 466, the same component may be used to determine whether the wearer is using the orthosis 300, whether the wearer is active or how active the wearer is, and whether the wearer is asleep.

The patient-responsive orthosis 300 may also include a force sensor 468. The force sensor 468 may be any suitable device that is capable of measuring the force exerted on the hand splint 362 by the hand or wrist of the wearer. Accordingly, the force sensor 468 measures an indication of the resistance of the patient's hand/wrist to the stretching.

The patient-responsive orthosis 300 may include a communications port 480 that enables data to be exported to an external device or, in some embodiments, allows the orthosis 300 to be controlled by another user (e.g., a parent or health care practitioner). In some embodiments, the communications port 480 may be a hardware port that enables wired communication (e.g., a universal serial bus port). In other embodiments, the communications port 480 may be a wireless communications module, such as a Bluetooth transceiver to send and receive short range wireless signals or a WiFi transceiver to join a local area network. (BLUETOOTH is a registered trademark of Bluetooth SIG, Inc.)

In some embodiments, the component package 400 may be integrated with the patient-responsive orthosis 300. In other embodiments, the component package 400 (and, in some instances, the motor 380) may be a separate package configured to couple and uncouple from the patient-responsive orthosis 300 or another patient-responsive orthosis.

The displacement sensor 462 enables the microcontroller 420 to accurately determine the position of the wearer's hand (i.e., how “stretched out” the wearer's hand is). The microcontroller 420 can then cause the motor 380 to gradually move the hand actuator 360 (e.g., rotate the hand splint 362 and translate the attachment mechanism 364) to extend the hand of the wearer. The microcontroller 420 may be programmed to extend the wearer's hand as a part of a standard treatment regimen or based on a treatment regimen developed specifically for the individual wearer. For instance, the angular rotation of the hand actuator 360 may be chosen by a clinician based on the range of motion of the wearer. Additionally, the frequency with which the hand actuator 360 is extended and the length of time that the hand actuator 360 remains extended may be based on a treatment schedule developed specifically for the individual wearer. The microcontroller 420 may be programmed to implement a progressive treatment schedule that, for example, gradually increases the angular movement of the hand actuator 360 and/or gradually increases the time that the hand actuator 360 is extended over time.

The communications port 480 enables the patient-responsive orthosis 300 to be accessed by a remote device (either wirelessly or via a wired connection) and controlled by a clinician and/or parent. For example, a clinician may access the patient-responsive orthosis 300 and set the set point of the orthosis 300 (e.g., the angle θ of the hand splint 362, relative to the angle of the forearm splint 340, when the hand actuator 360 is in the extended position), the time to the set point (i.e., the time period over which the motor 380 gradually extends the hand actuator 360), the time period that the hand actuator 360 should remain extended, the pattern of activation (i.e., when and how often the hand actuator 360 should extend). Because the microcontroller 420 may be programmed—and reprogrammed—to provide a custom treatment schedule, the patient-responsive orthosis 300 enables treatment for the patient to be adjusted over time without the need for a technician to manually adjust or refit a new orthosis.

As briefly mentioned above, the patient-responsive orthosis 300 may include a sleep sensor 464 that can output an indication to the microcontroller 420 that the wearer is asleep. Accordingly, the microcontroller 420 may be programmed to extend the hand actuator 360 only in response to a determination that the wearer is asleep (or has been asleep for a predetermined amount of time). The microcontroller 420 may cause the motor 380 to extend the hand actuator 360 gradually so as to reduce the likelihood that extending the wearer's hand will disturb the sleep of the wearer.

By allowing the wearer to fall asleep comfortably, the patient-responsive orthosis 300 improves the quality of life of the wearer and, because the wearer is likely to be a child, a caregiver for the wearer. Allowing the wearer to fall asleep comfortably may also improve patient adherence to the prescribed treatment regimen, which may be implemented only as the wearer is sleeping. Better adherence to the prescribed treatment regimen may improve clinical outcomes for patients. Additionally, because lack of adherence to treatment can disqualify a patient from surgical treatments, the patient-responsive orthosis 300 may help the wearer remain eligible for surgery.

The microcontroller 420 may be programmed to cause the motor 360 to extend the hand actuator 360 by a predetermined angle or distance (e.g., as determined by the microcontroller 420 based on the output of the displacement sensor 462). Additionally or alternatively, the microcontroller 420 may be programmed to cause the motor 360 to extend the hand actuator 360 until the hand or wrist of the user exerts a predetermined amount of resistance (as output by the force sensor 468). Therefore, the standard treatment regimen or treatment regimen developed specifically for the individual wearer may include extending the wearer's hand by a predetermined angle or distance or until the hand or wrist of the user exerts a predetermined amount of resistance.

The microcontroller 420 may store usage data in the memory 440. The usage data may include the amount of time the patient-responsive orthosis 300 is worn by the wearer (e.g., as determined by the microcontroller 420 based on the output of the use sensor 466), the time that the hand actuator 360 was extended (e.g., as determined by the microcontroller 420 based on the output of the displacement sensor 462), the displacement of the hand actuator 360 while extended (e.g., as determined by the microcontroller 420 based on the output of the displacement sensor 462), the sleep state of the wearer during the time that the hand actuator 360 was extended (e.g., as determined by the microcontroller 420 based on the output of the sleep sensor 464), the resistance of the patient's hand/wrist to the stretching (e.g., the force exerted by the hand or wrist on the hand splint 362 as determined by the force sensor 468), etc.

The communications port 480 enables the usage data described above to be exported to a remote device so a clinician, the wearer, and/or a caregiver may track the usage and progress of the wearer. Accordingly, the patient-responsive orthosis 300 enables a clinician or caregiver to better tailor the prescribed treatment for the individual wearer. The usage data may also be used as evidence of treatment, for example to facilitate payment from a third-party payor (e.g., an insurance company). Additionally, usage data from multiple patient-responsive orthoses 300 may be aggregated and analyzed to study the efficacy of various treatments.

Orthoses are generally either complete, generic, “off-the-shelf” devices or are custom-built by an orthotist or occupational therapist for a specific wearer. Due to the individualized treatment needed, custom orthoses are usually preferred by clinicians. When such devices are not accessible to patients, off-the-shelf devices are used.

The patient-responsive orthosis 300 improves both use cases. For all the reasons described above, a patient-responsive orthosis 300 that is custom-built for the wearer can provide treatment that is even more targeted to the individual clinical needs of the wearer than a prior art, custom-built orthosis. Meanwhile, the component package 400 (including the motor 380) can make even a generic, off-the-shelf orthosis responsive to the wearer and incorporate it into a treatment plan that is individually tailored to meet the specific clinical needs of the wearer. In some instances, complete patient-responsive orthoses 300 may be available in a variety of sizes. In other instances, the component package 400 may be removable and attachable to a variety of orthoses 300 (each including a fabric cuff 320, a forearm splint 340, and a hand actuator 360) that are available in a variety of sizes.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

What is claimed is:

1. An orthosis, comprising:
a forearm splint that is attachable to a forearm of a wearer;
a hand actuator that is attachable to the hand or fingers of the wearer;
a motor that extends the hand of the wearer by moving the hand actuator relative to the forearm splint from a relaxed position to an extended position;
memory that stores instructions to extend the hand of the wearer; and
a processor that extends the hand of the wearer in response to the instructions by causing the motor to move the hand actuator relative to the forearm splint.

2. The orthosis of claim 1, further comprising:
a sleep sensor that outputs an indication that the wearer is asleep to the processor,
wherein the processor extends the hand of the wearer in response to the instructions and an indication that the wearer is asleep.

3. The orthosis of claim 1, further comprising:
a communications port that enables the instructions to be specified by a user of a remote device and stored in the memory.

4. The orthosis of claim 3, wherein the instructions specified by the user of the remote device include a time that the hand actuator is moved from the relaxed position to the extended position, a frequency with which the hand actuator is moved from the relaxed position to the extended position, a time period over which the hand actuator is moved from the relaxed position to the extended position, or a time period over which the hand actuator remains in the extended position before moving to the relaxed position.

5. The orthosis of claim 3, wherein:
the orthosis further includes a displacement sensor that outputs an indication of the location of the hand actuator;
the displacement of the hand actuator between the relaxed position and the extended position is specified by the user of the remote device.

6. The orthosis of claim 3, wherein the processor stores usage data indicative of the use of the orthosis in the memory.

7. The orthosis of claim 6, wherein the communications port enables the usage data to be output to the remote device.

8. A method of extending a hand of a wearer of an orthosis having a forearm splint that is attachable to a forearm of a wearer, a hand actuator that is attachable to the hand or fingers of the wearer, and a motor that extends the hand of the wearer by moving the hand actuator relative to the forearm splint from a relaxed position to an extended position, the method comprising:
receiving instructions to extend the hand of the wearer;
storing the instructions in memory; and
extending the hand of the wearer, by a processor in response to the instructions, by causing the motor to move the hand actuator from the relaxed position to the extended position.

9. The method of claim 8, wherein:
the orthosis further includes a sleep sensor that outputs an indication that the wearer is asleep to the processor,
extending the hand of the wearer in response to the instructions further comprises extending the hand of the wearer in response to an indication that the wearer is asleep.

10. The method of claim 8, wherein:
the orthosis further includes a communications port; and
receiving the instructions comprises receiving instructions specified by a user of a remote device via the communications port.

11. The method of claim 10, wherein the instructions specified by the user of the remote device include a time that the hand actuator is moved from the relaxed position to the extended position, a frequency with which the hand actuator is moved from the relaxed position to the extended position, a time period over which the hand actuator is moved from the relaxed position to the extended position, or a time period over which the hand actuator remains in the extended position before moving to the relaxed position.

12. The method of claim 10, wherein:
the orthosis further includes a displacement sensor that outputs an indication of the location of the hand actuator;
the displacement of the hand actuator between the relaxed position and the extended position is specified by the user of the remote device.

13. The method of claim 10, further comprising:
storing usage data indicative of the use of the orthosis in the memory.

14. The method of claim 13, further comprising:
outputting the usage data to the remote device via the communications port.

15. A component package for an orthosis having a forearm splint that is attachable to a forearm of a wearer, a hand actuator that is attachable to the hand or fingers of the wearer, and a motor that extends the hand of the wearer by moving the hand actuator relative to the forearm splint from a relaxed position to an extended position, the component package comprising:
memory that stores instructions to extend the hand of the wearer; and
a processor that extends the hand of the wearer in response to the instructions by causing the motor to move the hand actuator relative to the forearm splint.

16. The component package of claim 15, further comprising:
a sleep sensor that outputs an indication that the wearer is asleep to the processor,
wherein the processor extends the hand of the wearer in response to the instructions and an indication that the wearer is asleep.

17. The component package of claim 15, further comprising:
a communications port that enables the instructions to be specified by a user of a remote device and stored in the memory.

18. The component package of claim 15, wherein the instructions specified by the user of the remote device include a time that the hand actuator is moved from the relaxed position to the extended position, a frequency with which the hand actuator is moved from the relaxed position to the extended position, a time period over which the hand actuator is moved from the relaxed position to the extended position, or a time period over which the hand actuator remains in the extended position before moving to the relaxed position.

19. The component package of claim 15, further comprising:
a displacement sensor that outputs an indication of the location of the hand actuator,
wherein the displacement of the hand actuator between the relaxed position and the extended position is specified by the user of the remote device.

20. The component package of claim 15, wherein:
the processor stores usage data indicative of the use of the orthosis in the memory; and
the communications port enables the usage data to be output to the remote device.
